(19) United States
(12) Patent Application Publication
(10) Pub. No.: US 2014/0030260 A1
(43) Pub. Date: Jan. 30, 2014

(54) METHODS AND COMPOSITIONS TO ELIMINATE CHRONIC LYMPHOCYTIC LEUKEMIA AND OTHER HEMATOLOGIC MALIGNANT CELLS IN STROMAL MICROENVIRONMENT FOR CANCER THERAPY

(71) Applicants: Peng Huang, Bellaire, TX (US); Wan Zhang, Houston, TX (US); Michael J. Keating, Houston, TX (US)

(72) Inventors: Peng Huang, Bellaire, TX (US); Wan Zhang, Houston, TX (US); Michael J. Keating, Houston, TX (US)

(73) Assignee: BOARD OF REGENTS, THE UNIVERSITY OF TEXAS SYSTEM, Austin, TX (US)

(21) Appl. No.: 13/989,016

(22) PCT Filed: Nov. 22, 2011
(86) PCT No.: PCT/US2011/061788

§ 371 Date: Oct. 11, 2013

Related U.S. Application Data
(60) Provisional application No. 61/416,073, filed on Nov. 22, 2010.

(51) Int. Cl. A61K 31/195; A61K 33/20; A61K 45/06; A61K 31/095

(52) U.S. Cl. CPC A61K 31/195; A61K 31/095; A61K 33/20; A61K 45/06
USPC 424/133.1; 514/561; 424/93.7; 514/110; 514/48; 514/492; 514/394; 424/142.1; 514/323

(57) ABSTRACT

The present invention regards compositions and methods for treating chronic lymphocytic leukemia in the in vivo tissue environment using selenium-containing compositions. In particular aspects, a selenium-comprising compound is administered to an individual wherein the CLL cells are in a stromal cell environment, wherein stromal factors modulate the selenium comprising compound to enhance its activity.

This application claims priority to U.S. Provisional Patent Application Ser. No. 61/416,073, filed Nov. 22, 2010, which is incorporated by reference herein in its entirety.

### TECHNICAL FIELD

The present invention generally concerns the fields of medicine, molecular biology, and cell biology. In particular, the present invention concerns cancer therapy of hematologic malignant cells, including chronic lymphocytic leukemia cells, using selenium-containing compositions.

### BACKGROUND OF THE INVENTION

Development of drug resistance in cancer cells represents a major challenge in the clinical treatment of cancer. Compelling evidence indicates that the tumor stromal microenvironment plays a major role in promoting cancer cell survival and therapeutic resistance. For instance, stromal cells provide a significant survival signal to leukemia cells leading to drug resistance, and this is especially obvious in chronic lymphocytic leukemia (CLL). The fact that CLL cells have a prolonged survival time and abnormally accumulate in vivo but are prone to undergo apoptosis the in vitro (cell culture) indicates the importance of in vivo microenvironment to sustain the survival of CLL cells. These observations highlight the difficulty in eliminating leukemia cells in vivo in which the malignant cells are protected by the stromal environment. Thus, it is extremely important to develop agents that can effectively kill cancer cells under the stromal protection.

### BRIEF SUMMARY OF THE INVENTION

The present invention is generally directed to methods and compositions for the treatment of cancer. In particular aspects, the invention concerns treatment of cancers and in particular of cancers under stromal protection. In certain cases, the invention concerns treatment of cancers that are protected from the cytotoxic effect of chemotherapeutic agents by stromal cells.

In embodiments of the present invention, an exemplary selenium-containing compound, known as selenocystine, can preferentially kill primary leukemia cells isolated from CLL patients in the presence of stromal cells. Strikingly, this compound exhibited only moderate cytotoxic effect on leukemia cells when they were cultured alone, but caused considerable leukemia cell death in the presence of stromal cells. It was determined that a secreted factor from stromal cells can convert selenocystine to active compounds that are preferentially toxic to CLL cells. However, in certain cases the active compound is chemically unstable and thus difficult to manufacture and store. To overcome this, the inventors developed novel drug compositions, designated as selecticines, that can be readily prepared and used to selectively kill cancer cells, such as leukemia cells, and overcome drug resistance in stromal environment. The inventors demonstrated that an active component of selecticines at the micromolar concentration range was very effective in killing primary CLL cells, for example, in the presence of various types of stromal cells. Importantly, this compound exhibited low toxicity to normal cells even in the stromal environment. Furthermore, its combination with the standard but merely exemplary chemotherapeutic agent fludarabine showed synergistic effect against CLL cells, indicating its significant utility in drug combination for leukemia treatment. Because leukemia cells and solid tumor cells are always under the protection of stromal tissue environment in vivo and as such are resistant to conventional chemotherapeutic agents, the drug compositions described in this invention have significant therapeutic applications in clinical treatment of cancer, owing to their ability to effectively kill cancer cells in the presence of stromal cells.

In one embodiment of the present invention, there is a method of treating chronic lymphocytic leukemia in an individual, comprising the step of administering a therapeutically effective amount of a selecticine to the individual. In specific cases, the selecticine is selected from the group consisting of selenocystine (which may be referred to as 2-amino-3-(2-amino-2-carboxy-ethyl)selanylselanyl-propanoic acid), selenocysteine (which may be referred to as 3-selenyl-2-aminopropanoic acid or selenium cysteine), a combination of selenocystine and 2-mercaptoethanol, a combination of selenocystine and HCL, and a combination of selenocystine and dithiothreitol.

In certain embodiments of the invention, there are compositions of selecticine and methods to prepare selecticine. In specific cases, the invention encompasses methods of using selecticine to kill chronic lymphocytic leukemia (CLL) cells in a stromal tissue microenvironment for treatment of CLL patients. In certain cases, the invention encompasses methods of using selecticine to kill malignant B cells in a stromal tissue microenvironment for treatment of B cell malignancies, such as B cell lymphoma and multiple myeloma, for example. In specific embodiments, the present invention concerns the use of selecticine in combination with other anticancer agents to kill chronic lymphocytic leukemia (CLL) cells in a stromal tissue microenvironment for treatment of CLL patients. In some embodiments, the present invention regards the use of selecticine in combination with other anticancer agents to kill malignant B cells in tissue stromal microenvironment for treatment of other B cell malignancies, such as B cell lymphoma and multiple myeloma. In particular aspects, the invention concerns the use of selecticine to kill acute myeloid leukemia (AML) cells in tissue stromal microenvironment for treatment of patients with AML and other hematologic malignancies. In certain cases, the invention includes the use of selecticine in combination with other drugs to kill acute myeloid leukemia (AML) cells in tissue stromal microenvironment for treatment of patients with AML and other hematologic malignancies.

In some embodiments of the invention, methods of the invention further comprises administering in conjunction with the present invention an additional anti-cancer therapy to the individual, such as chemotherapy, immunotherapy, surgery, stem cell transplant, or radiation, for example. In specific embodiments for chemotherapy, it comprises one or more of fludarabine, cyclophosphamide, bendamustine, and/or oxaliplatin. In particular cases where the method is employed in conjunction with immunotherapy, the immunotherapy may comprise rituximab, alemtuzumab, ofatumumab, and/or lumiliximab. In particular embodiments, the selecticine and the additional cancer therapy, such as the chemotherapy or immunotherapy, for example, work synergistically.

In some cases, the compositions of the present invention are delivered to the individual in need thereof before another anti-cancer therapy. The timing of the delivery may be of any suitable regimen, although in specific cases the present invention is delivered one or more years before the other anti-cancer therapy, one or more months, one or more weeks, one or more days, or one or more hours before the other anti-cancer therapy. The selecticine composition may be delivered six months, five months, four months, three months, two months, one month, three weeks, two weeks, one week, six days, five days, four days, three days, two days, one day, 18 hours, 12 hours, 8 hours, 6 hours, 4 hours, 3 hours, 2 hours, or one hour before the other anti-cancer therapy.

In some cases, the compositions of the present invention are delivered to the individual in need thereof after another anti-cancer therapy. The timing of the delivery may be of any suitable regimen, although in specific cases the present invention is delivered one or more years after the other anti-cancer therapy, one or more months, one or more weeks, one or more days, or one or more hours before the other anti-cancer therapy. The selecticine composition may be delivered six months, five months, four months, three months, two months, one month, three weeks, two weeks, one week, six days, five days, four days, three days, two days, one day, 18 hours, 12 hours, 8 hours, 6 hours, 4 hours, 3 hours, 2 hours, or one hour after the other anti-cancer therapy.

Administration of the present inventive compositions to the individual in need thereof may be by any suitable means, but in specific embodiments the compositions are delivered systemically or locally. In specific embodiments, the administration is performed intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, or in lipid compositions (e.g, liposomes).

Kits for therapy of hematologic malignancies are encompassed by the invention. The kit may include therapeutic compositions of the invention, including one or more components of a selecticine composition of the invention. In specific cases the kit further includes an additional anti-cancer therapeutic agent and/or a means for delivering a therapeutic composition.

The individual being treated with methods of the invention include mammals, such as humans, monkeys, dogs, cats, cows, horses, pigs, goats, and sheep.

In an embodiment of the present invention, there is a method of treating chronic lymphocytic leukemia in an individual, comprising the step of administering a therapeutically effective amount of a selecticine to the individual. In a specific aspect, the selecticine is selected from the group consisting of selenocystine, selenocysteine, a combination of selenocystine and 2-mercaptoethanol, a combination of selenocystine and dithiothreitol, and a combination of selenocystine and hydrochloric acid. In particular cases, the selecticine is selonocystine or selenocysteine. In some aspects, the selecticine is a combination of selenocystine and 2-mercaptoethanol; selenocystine and hydrochloric acid; or selenocystine and dithiothreitol.

In one embodiment of the invention, methods described herein further comprise administering an additional anti-cancer therapy to the individual. In a specific embodiment, the anti-cancer therapy comprises chemotherapy, immunotherapy, surgery, stem cell transplant, or radiation. In specific aspects, the chemotherapy comprises fludarabine, cyclophosphamide, bendamustine and/or oxaliplatin. In some embodiments, the immunotherapy comprises rituximab, alemtuzumab, ofatumumab, cyclophosphamide, lumiliximab, and/or lenalidomide.

In one embodiment of the invention, the selecticine is delivered to the individual intravenously or orally.

In a particular embodiment of the invention, the method further comprises the step of diagnosing chronic lymphocytic leukemia, such as by blood assay, pathology, bone marrow biopsy, ultrasound, MRI, CT scan, and so forth.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 illustrates exemplary chemical structures of certain components of some aspects of the invention.

FIG. 2 shows a specific embodiment of chemical composition of selecticine (referred to herein as selecticine-01) and method of preparation.

FIG. 3 shows a specific embodiment of chemical composition of selecticine (referred to herein as selecticine-02).

FIG. 4 shows a specific embodiment of chemical composition of selecticine (referred to herein as selecticine-03).

FIG. 5 demonstrates selective killing of primary leukemia cells by selecticine in the presence of stromal cells (patient 1).

FIG. 6 shows selective killing of primary leukemia cells (CLL) by selecticine in the presence of stromal cells.

FIG. 7 demonstrates selective killing of primary leukemia cells (CLL) by selecticine in the presence of stromal cells (patient 3).

FIG. 8 shows selecticine exhibits only moderate cytotoxicity to immortalized normal lymphocytes in the presence of stromal cells.

FIG. 9 provides evidence of selective killing of primary leukemia cells (CLL) by selecticine in the presence of different types of stromal cells.

FIG. 10 provides comparison of cytotoxicity of selenocystine, F-ara-A (active form of fludarabine), and oxaliplatin in CLL cells, cultured alone or in the presence of different types of stromal cells (patient 4).

FIG. 11 shows enhancement of cytotoxic action of fludarabine (F-ara-A) and oxaliplatine by selecticine in CLL cells co-cultured with stroma cells (patient 5).

FIG. 12 shows that there is a stromal factor in the stromal cell culture medium (CM, conditioned medium) that can converted selenocystine into an active compound highly toxic to CLL cells.

FIG. 13 illustrates exemplary procedures to identify a low-molecular-weight factor that converts selenocystine to an active compound selectively toxic to CLL cells.

FIG. 14 shows that selecticine-02 is highly toxic to CLL cells even in the absence of stroma cells.

FIG. 15 demonstrates the molar ration of selenocystine:2-mercaptoethanol is critical for the cytotoxic activity of Selecticine-02.

FIG. 16 shows that selenocystine exhibits moderate cytotoxicity in human promyelocytic leukemia cells (HL-60) and the presence or absence of bone marrow stromal cells.

FIG. 17 demonstrates that selecticine exhibits cytotoxic effect on human B cell lymphoma cells (Raji) and stromal cells further increased this cytotoxic effect.

FIG. 18 demonstrates selective killing of primary leukemia cells (CLL) from multiple patient samples by selecticine in the presence of stromal cells.

FIG. 19 shows that selecticine enhances the cytotoxicity of fludarabine and oxaliplatine in CLL cells co-cultured with HS-5 stromal cells.

FIG. 20 shows selecticine enhances the cytotoxicity of fludarabine and oxaliplatine in CLL cells co-cultured with NKtert stromal cells.

FIG. 21 shows selecticine enhances the cytotoxicity of fludarabine and oxaliplatine in CLL cells co-cultured with KUSA stromal cells.

FIG. 22 shows that selecticine promotes ROS generation and causes depletion of cellular thiol in CLL cells in the presence of stromal cells.

FIG. 23 shows that catalase abrogated the cytotoxic effect of selecticine in CLL cells under stromal co-culture conditions.

FIG. 24 shows that SOD and catalase protect CLL cells from the cytotoxic effect of selecticine in the presence of stromal cells.

FIG. 25 shows the effect of antioxidant NAC and the glutathione-depleting agent BSO on the cytotoxic effect of selecticine in the presence of stromal cells.

FIG. 26 shows a stromal factor secreted in the stromal conditioned medium (CM) can convert selecticine into an active compound that is toxic to CLL cells.

FIG. 27 illustrates exemplary procedures to identify a low-molecular-weight factor that converts selecticine to an active compound selectively toxic to CLL cells.

FIG. 28 shows selecticine-02 is highly toxic to CLL cells, even in the absence of stroma cells.

FIG. 29 demonstrates the ration of selenocystine: -mercaptoethanol is critical for the activity of selecticine-02.

FIG. 30 shows that selecticine-02 with a ration of 1:5 (selenocystine: -mercaptoethanol) was highly effective against B-cell lymphoma.

FIG. 31 demonstrates that selecticine effectively killed primary leukemia cells from a patients with acute myeloid leukemia (AML) in the presence of bone marrow stromal cells.

FIG. 32 presents in vivo therapeutic activity of selecticine in CLL mouse model.

FIG. 33 shows the effect of selecticine treatment on mouse body weights.

### DETAILED DESCRIPTION OF THE INVENTION

In the following description, certain details are set forth such as specific quantities, sizes, etc. so as to provide a thorough understanding of the present embodiments disclosed herein. However, it will be obvious to those skilled in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

### I. Definitions

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

The term “selecticine” as used herein refers to chemical compositions comprising selenocystine or selenocystiene and agents that make these selenium-containing compounds soluble and stable, thereby suitable for therapeutic applications. Examples of such agents include hydrochloric acid, 2-mercaptoethanol, and dithiothreitol.

As used herein, “therapeutically effective amount” refers to the amount of a compound that, when administered to a mammal for treating a state, disorder or condition, is sufficient to effect such treatment. The “therapeutically effective amount” will vary depending on the compound, the disease and its severity, and the age, weight, physical condition and responsiveness of the mammal to be treated. As used herein the term “therapeutically effective amount” refers to an amount of a compound sufficient to prevent, inhibit, reduce, or eliminate one or more causes, symptoms, or complications of cancer, including leukemia, for example chronic lymphocytic leukemia.

As used herein, “treating” or “treatment” of a state, disorder or condition includes: (1) preventing or delaying the appearance of clinical symptoms of the state, disorder or condition developing in a mammal that may be afflicted with or predisposed to the state, disorder or condition, but does not yet experience or display clinical or subclinical symptoms of the state, disorder or condition; (2) inhibiting the state, disorder or condition, i.e., arresting or reducing the development of the disease or at least one clinical or subclinical symptom thereof; or (3) relieving the disease, i.e., causing regression of the state, disorder or condition or at least one of its clinical or subclinical symptoms.

### II. Exemplary Embodiments of the Invention

The present invention, in certain aspects, generally concerns cancer-stromal interaction. In particular aspects concerning cancer-stromal cell interaction, the selenium-containing compound selenocystine, when properly prepared and used, exhibits a highly potent cytotoxic effect against chronic lymphocytic leukemia (CLL) cells isolated from the blood samples of CLL patients and effectively kills the leukemia cells in the presence of bone marrow stromal cells through a reactive oxygen species (ROS)-mediated mechanism. CLL cells are intrinsically under oxidative stress due to high basal ROS generation. The bone marrow stromal cells promote glutathione synthesis in the CLL cells and thus help the leukemia cells to maintain redox balance and promote cell viability. However, in the presence of properly solubilized selenocystine, the stromal cells convert selenocystine to a highly active compound that induces a significant increase in ROS production in the CLL cells, leading to severe oxidative damage to the leukemia cells. Importantly, selenocystine showed only moderate cytotoxic effect toward leukemia cells in the absence of stromal cells, indicating that the presence of stromal cells promotes the cytotoxicity of this compound against the leukemia cells and thus selenocystine has high therapeutic effect in vivo, where stromal cells are constantly present, in particular embodiments of the invention. These novel observations led to the investigation of the underlying mechanism and the development of methods and compositions for treatment of chronic lymphocytic leukemia and other B cell malignancies.

Mechanistic studies revealed that the bone marrow stromal cells convert selenocystine to a low molecular weight component (selenocysteine) as an active compound that is highly toxic to CLL leukemia cells with low toxicity to normal cells. Because the active component selenocysteine is chemically unstable and unsuitable to be stored and used as a anticancer drug, the present invention encompasses methods to prepare stable compositions for use in the treatment of lymphocytic leukemia and other B cell malignancies in vivo.

In other embodiments of the invention, it is demonstrated herein that selecticine is effective in killing primary leukemia cells isolated from patients with acute myeloid leukemia (AML) in the presence of bone marrow stromal cells. In addition, an exemplary animal study using a CLL disease mouse model demonstrated that selecticine is highly effective in vivo and significantly prolonged the survival time of mice with the B-cell leukemia.

In particular embodiments of the invention, the methods and compositions have the ability to overcome drug resistance due to stromal protection. In certain cases, there is a synergistic anticancer effect when selecticine is combined with standard chemotherapeutic agents.

In specific embodiments, the selecticine has the ability to kill leukemia cells in vivo regardless of whether the leukemia cells are in close contact with the stromal cells, or in the tissue microenvironment where the leukemia cells have left their bone marrow tissue niches and are not in direct contact with the bone marrow stromal cells. Under the later conditions, selecticine specifically in its active form (designated as selecticine-02) is able to kill the leukemia cells effectively.

### III. Certain Compositions of the Invention

In some cases, the selecticine composition comprises two or more compounds, and in specific aspects at least one of the multiple components of the selecticine facilitates conversion of selenocystine to selenocysteine. In specific embodiments, one of the components of the selecticine allows or facilitates modulation of a form of a compound that is not able to be transported into the cancer cell into a form of compound that is able to be transported into the cancer cells (primary leukemia cells from CLL patients). In specific embodiments, the compound that facilitates conversion of selenocystine to selenocysteine is the same compound that facilitates modulation of a form of a compound that is not able to be transported into the CLL cells into a form of compound that is able to be transported into the CLL cells. Although in specific cases any component that facilitates conversion of selenocystine to selenocysteine is provided in vivo by stromal cells in the cancer cell environment, in specific embodiments the compound that facilitates such conversion is delivered with the other component(s) of the selecticine.

In certain aspects the selecticine composition comprises selenocystine and a second compound. In particular embodiments of the invention, a certain ratio of two components of a composition of the invention, for example of selenocystine and a second compound is utilized. In specific cases, the ratio of selenocystine and second compound is 1:1, 1:1.5, 1:2, 1:3, 1:4, 1:5, 1:6, 1:7, 1:8, 1:9, 1:10, and so forth.

### IV. Chronic Lymphocytic Leukemia (CLL)

CLL symptoms usually develop slowly, and some cases are asymptomatic. Symptoms of CLL include fatigue, shortness of breath during normal activities, swollen lymph nodes or spleen, infections, weight loss, aches, fever or night sweats, for example.

The diagnosis of CLL is usually determined from blood and/or bone marrow tests. Blood tests for CLL include blood cell counts and a blood cell examination. An individual with CLL will have a high lymphocyte count and may also have a low red cell count and a low platelet count. For blood cell examination, flow cytometry or immunophenotyping may be employed to determine if CLL is the reason for the high lymphocyte count. Flow cytometry also illustrates if the CLL is B-cell CLL or T-Cell CLL, with B-cell CLL being the most prevalent. Immunoglobulin levels in the blood may also be determined as part of the diagnosis, given that individuals with CLL may have low levels of immunoglobulins. FISH may be used to see if there are changes to the chromosomes of the CLL cells, with sample cells coming from blood or marrow, and the results may give the health care provider information about treatment regimens. Computerized tomography (CT) may be used for diagnosis, in certain instances.

The treatment plan for a person with CLL depends on a variety of factors, including stage of the CLL (low, intermediate or high risk), physical exam and lab test results, overall health of the patient and, in some cases, the patient's age. The disease may be staged using the “Rai staging system” that defines a person's risk. In low-risk CLL, there is high lymphocyte count in the blood and the marrow; in intermediate-risk CLL, there is high lymphocyte count in the blood and the marrow and enlarged lymph nodes, liver or spleen. In high-risk CLL, there is high lymphocyte count in the blood and the marrow and anemia or a low platelet count. Other information can be obtained from the blood lymphocyte doubling time and level of beta 2-microglobulin. Risk factors for an individual that may develop CLL includes age (over 50), sex (men are more common), race (Caucasians are more common), family history of blood or bone marrow cancers, and exposure to chemicals, such as certain herbicides and insecticides.

In some cases, treatment for CLL can commence if one or more of the following indicators develop: the number of CLL cells is much higher than previously, the number of normal cells is much lower than previously, or the spleen or the lymph nodes have enlarged. Individuals that have intermediate- and higher-risk (faster-growing) CLL may be treated with one or more of chemotherapy, immunotherapy, radiation, surgery, stem cell transplantation, and/or splenectomy. Exemplary chemotherapy drugs include fludarabine, cyclophosphamide, lenalidomide, bendamustine, flavopiridol, oxaliplatin, and other conventional chemotherapies. Immunotherapy such as monoclonal antibody therapies include, for example, rituximab, alemtuzumab, Ofatumumab, and Lumiliximab.

A combination of chemotherapy and immunotherapy may be employed for the individual, including, for example, fludarabine with cyclophosphamide, oxaliplatin and/or rituximab. Bendamustine may be combined with rituximab or lenalidomide, in certain cases.

In addition to anti-cancer chemotherapy, the individual may be administered antibiotics, immunoglobulins, blood transfusions, red cell growth factors (such as darbepoetin alfa and epoetin alfa), compounds for palliative care, growth factors such as G-CSF or GM-CSF, for example.

The present invention may be employed as an initial treatment and/or following an initial treatment. The individuals being treated may be having therapy for the first time, be in remission, be complete responders, be partial responders, be non-responders to other drugs, have stable disease, have progressive disease, or have minimal residual disease, for example.

### V. Selenocysteine

Selenocysteine is not coded for directly in the genetic code, in contrast to other amino acids, but instead is encoded by a UGA codon, which is primarily a stop codon. The UGA codon is made to encode selenocysteine with the presence of a SElenoCysteine Insertion Sequence (SECIS element) in the mRNA. In eukaryotes and in archaeabacteria, the SECIS element is in the 3′ untranslated region (3′ UTR) of the mRNA and is able to direct multiple UGA codons to encode selenocysteine residues. In bacteria, however, the SECIS element is located immediately following the UGA codon within the reading frame for the selenoprotein. Selenophosphate is the selenium donor for synthesis of selenocysteyl-tRNA is, which is a reactive oxygen-labile compound.

No free pool of selenocysteine exists in the cell, because its high reactivity would incur damage to cells. As an alternative, cells store selenium in the selenide form (H2Se), which is less reactive.

### VI. Pharmaceutical Preparations

Pharmaceutical compositions of the present invention comprise an effective amount of one or more selecticine and, in some cases, an additional agent, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one selecticine will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The selecticine may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g, liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The selecticine may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include selecticine, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the selecticine may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 0.1% to about 20% of the weight of the unit, or between about 0.2% to about 2%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### A. Alimentary Compositions and Formulations

In some embodiments of the present invention, the selecticine is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft- shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally- administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### B. Parenteral Compositions and Formulations

In further embodiments, selecticine may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### C. Miscellaneous Pharmaceutical Compositions and Formulations

In other preferred embodiments of the invention, the active compound selecticine may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

The skilled artisan can consult Remington's Pharmaceutical Sciences, 18th Ed. Mack Printing Company, 1990, incorporated by reference herein in its entirety, for information about pharmaceutical compositions and delivery.

### VII. Exemplary Kits of the Invention

Any of the compositions described herein may be comprised in a kit. In a non-limiting example, a selecticine and, optionally an additional agent, may be comprised in a kit. The kits will thus comprise, in suitable container means, a selecticine and optionally an additional agent of the present invention.

The kits may comprise a suitably aliquoted selecticine and optional additional agent compositions of the present invention. The component(s) of the kits may be packaged either in aqueous media or in lyophilized form. The container means of the kits will generally include at least one vial, test tube, flask, bottle, syringe or other container means, into which a component may be placed, and suitably aliquoted, in specific cases. Where there are more than one component in the kit, the kit also will generally contain a second, third or other additional container into which the additional components may be separately placed. However, various combinations of components may be comprised in a vial. The kits of the present invention also will typically include a means for containing the selecticine, lipid, additional agent, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. The selecticine compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the invention may also comprise, and/or be packaged with, an instrument for assisting with the injection/administration and/or placement of the selecticine within the body of an animal. Such an instrument may be a syringe, pipette, forceps, and/or any such medically approved delivery vehicle, for example.

In some kits of the invention, there are reagents and/or devices that enable detection of cancer, including detection of hematologic malignancies, such as CLL.

### VIII. EXAMPLES

The following examples are presented in order to more fully illustrate the preferred embodiments of the invention. They should in no way, however, be construed as limiting the broad scope of the invention.

### Example 1

### Use of Selecticines and Active Components to Selectively Target Cancer Cells in Stromal Microenvironment

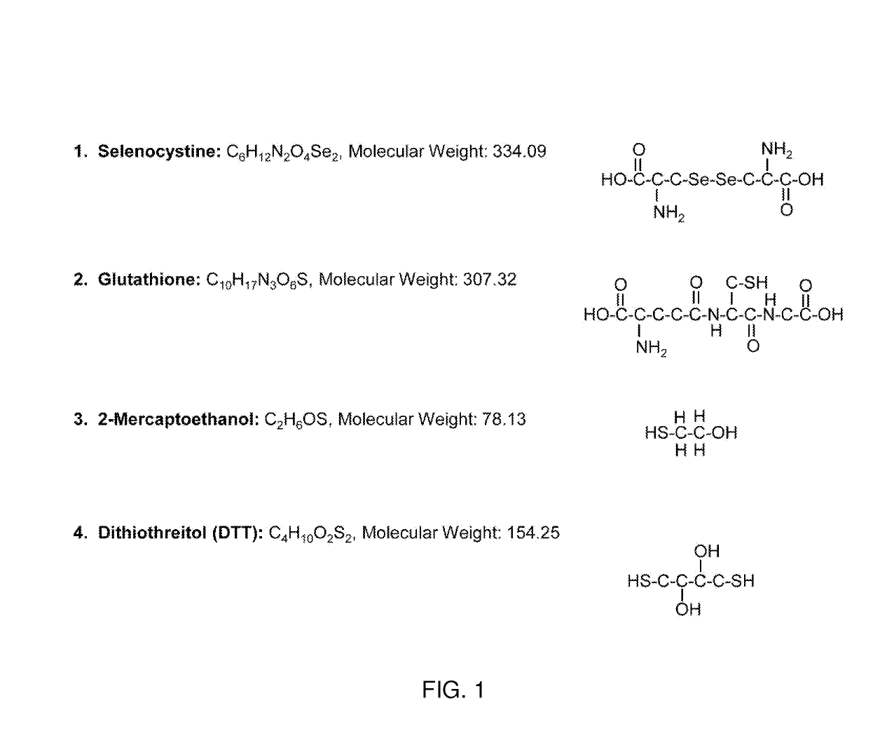

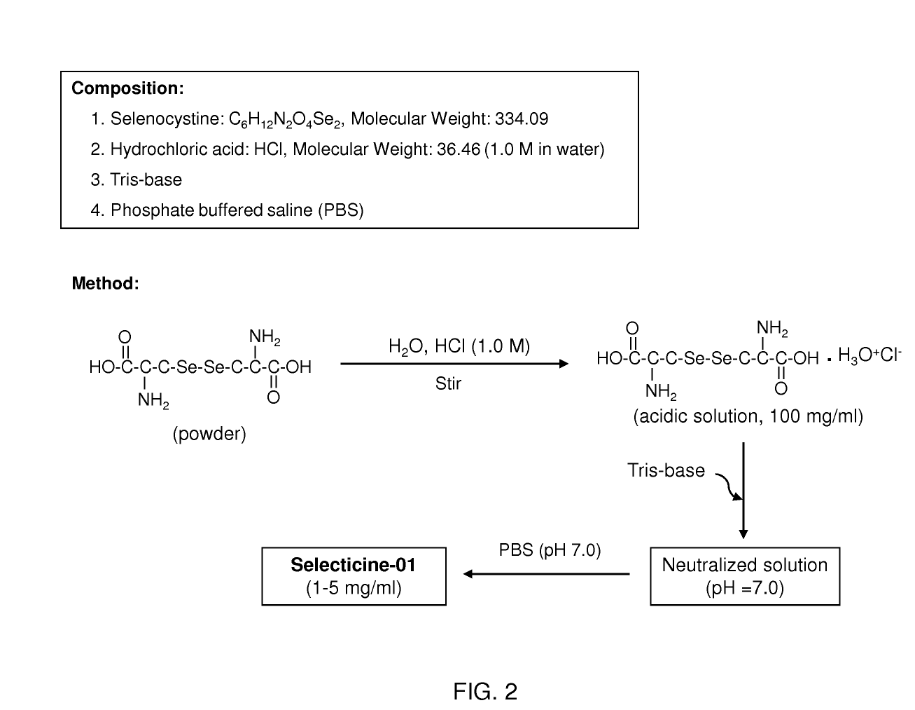

FIG. 1 illustrates exemplary chemical structures of certain components of some aspects of the invention. FIG. 2 shows a specific embodiment of chemical composition of selecticine and method for its preparation, which includes the use of 1.0 M HCl to solublize selenocystine, the use of Tris-base to neutralize the solution, and dilution of this solution with PBS to appropriate final concentrations.

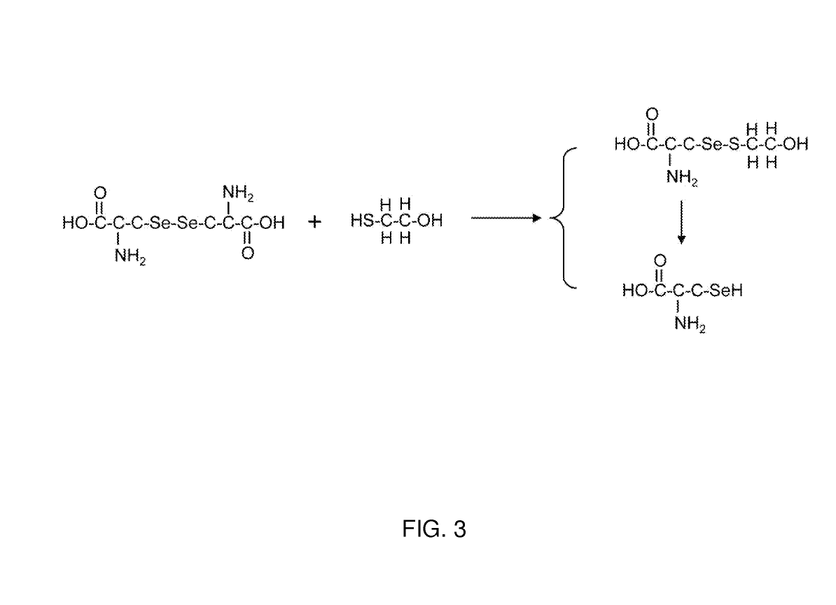

FIG. 3 shows a specific embodiment of chemical composition of selecticine-02. Exemplary compositions and conditions include 1 mole of selenocystine and 2-5 moles of 2-mercaptoethanol. The molar ratio of selenocystine and 2-mercaptoethanol is important in determining the anticancer activity of this particular selecticine. It is stored at 4° C.

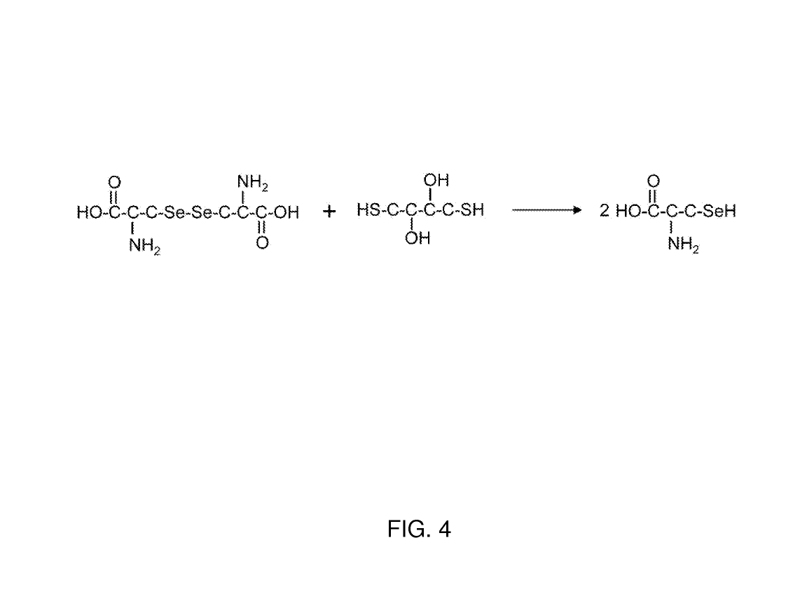

FIG. 4 shows a specific embodiment of chemical composition of selecticine-03. Exemplary compositions and conditions include 1 mole of selenocystine and 1 mole of dithiothreitol. PBS (pH 7.4) is mixed in, and it is stored at 4° C.

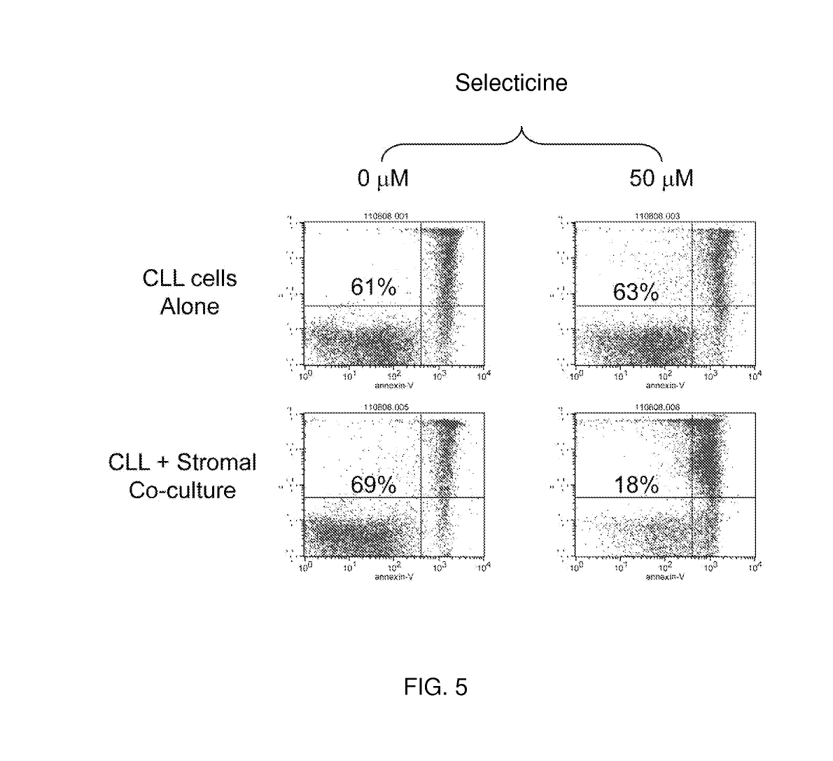

FIG. 5 demonstrates selective killing of primary leukemia cells by selecticine in the presence of stromal cells. Primary leukemia cells were isolated from CLL patient #1, and cultured in vitro alone or in the presence of human bone marrow cells (HS-5 cell line) as indicated. The cells were then treated with 50 μM selecticine for 2 days, and cell viability was measured by flow cytometry analysis after double-staining with annexin-V and PI. The numbers indicate % of viable cells.

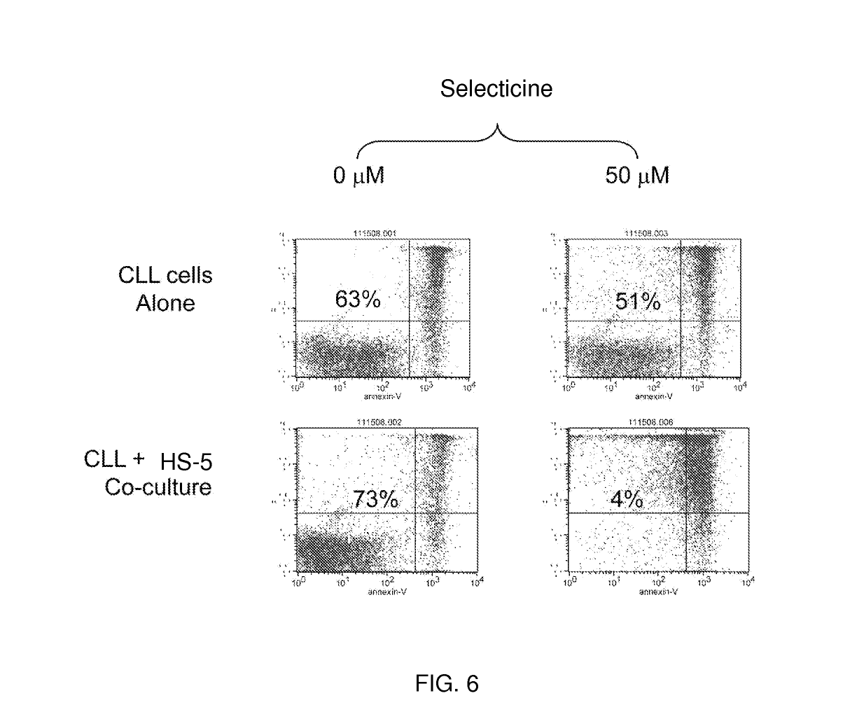

FIG. 6 shows selective killing of primary leukemia cells (CLL) by selecticine in the presence of stromal cells. CLL cells were isolated from patient #2, and cultured in vitro alone or in the presence of human bone marrow stromal cells (HS-5 cell line) as indicated. The cells were then treated with 50 μM selecticine for 2 days, and cell viability was measured by flow cytometry analysis after double-staining with annexin-V and PI. The numbers indicate % of viable cells.

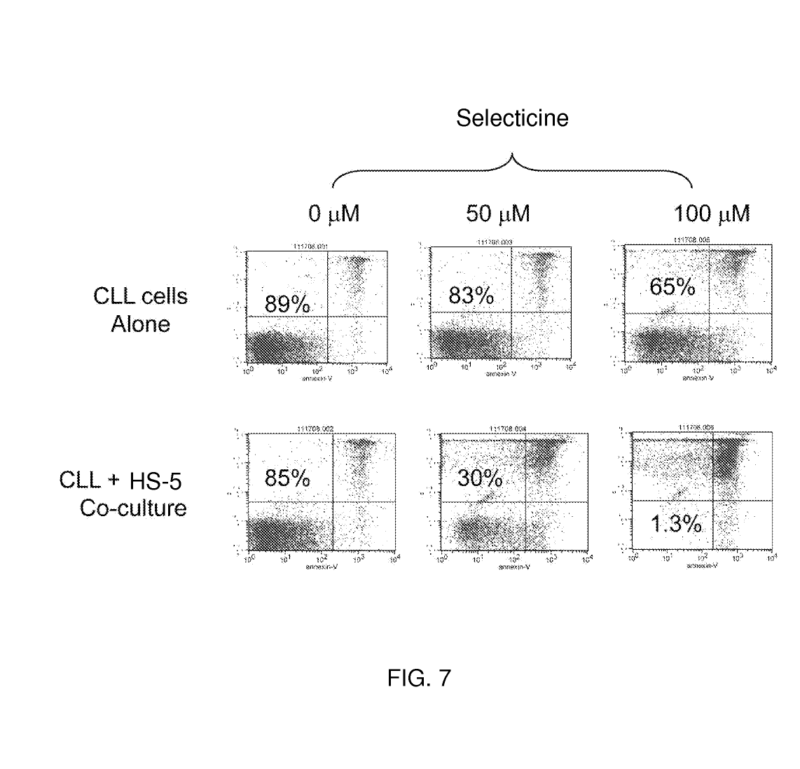

FIG. 7 demonstrates selective killing of primary leukemia cells (CLL) by selecticine in the presence of stromal cells. CLL cells were isolated from patient #3, and cultured in vitro alone or in the presence of human bone marrow stromal cells (HS-5 cells) as indicated. The cells were then treated with 50 or 100 μM selecticine for 2 days. Cell viability was then measured by flow cytometry analysis after double-staining with annexin-V and PI. The numbers indicate % of viable cells.

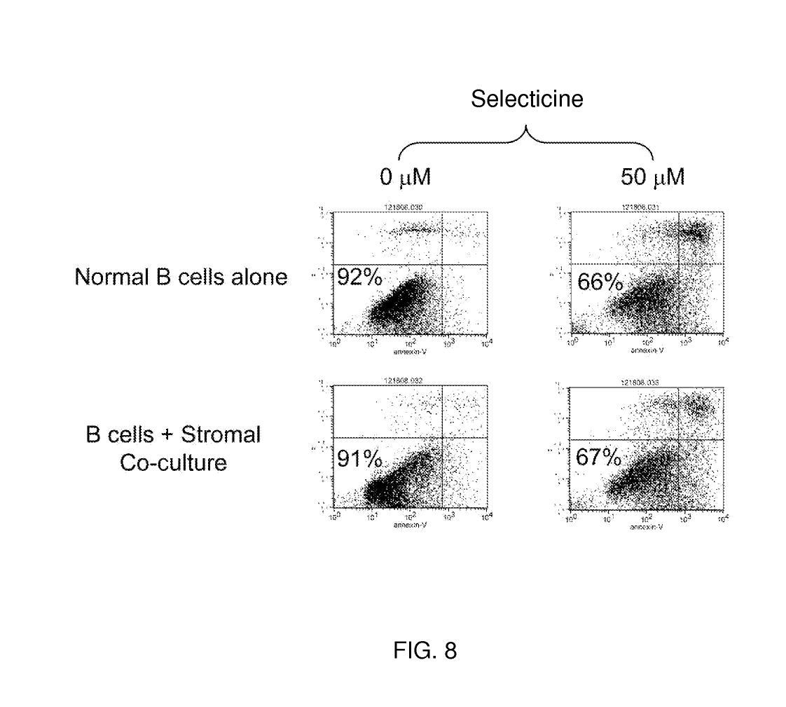

FIG. 8 shows selecticine exhibits only moderate cytotoxicity to immortalized normal lymphocytes in the presence of stromal cells. EBV-immortalized normal B-CLL lymphocytes were cultured alone or in the presence of human bone marrow stromal cells (HS-5 cell line) as indicated. The cells were then treated with 50 μM selecticine for 2 days, and cell viability was measured by flow cytometry analysis after double-staining with annexin-V and PI. The numbers indicate % of viable cells.

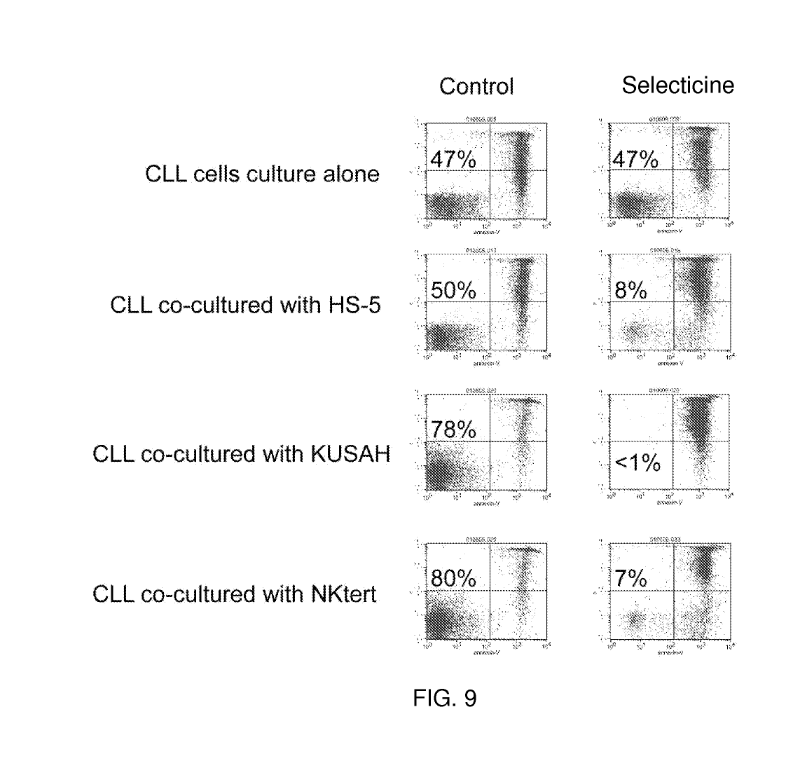

FIG. 9 provides evidence of selective killing of primary leukemia cells (CLL) by selecticine in the presence of different types of stromal cells (HS-5, KUSAH1, NKtert). CLL cells were isolated from the blood sample of a CLL patient (#9) and cultured in vitro alone or in the presence of different types of stromal cells as indicated. The cells were then treated with 50 μM selecticine for 2 days. Cell viability was then measured by flow cytometry analysis after double-staining with annexin-V and PI. The numbers indicate % of viable cells.

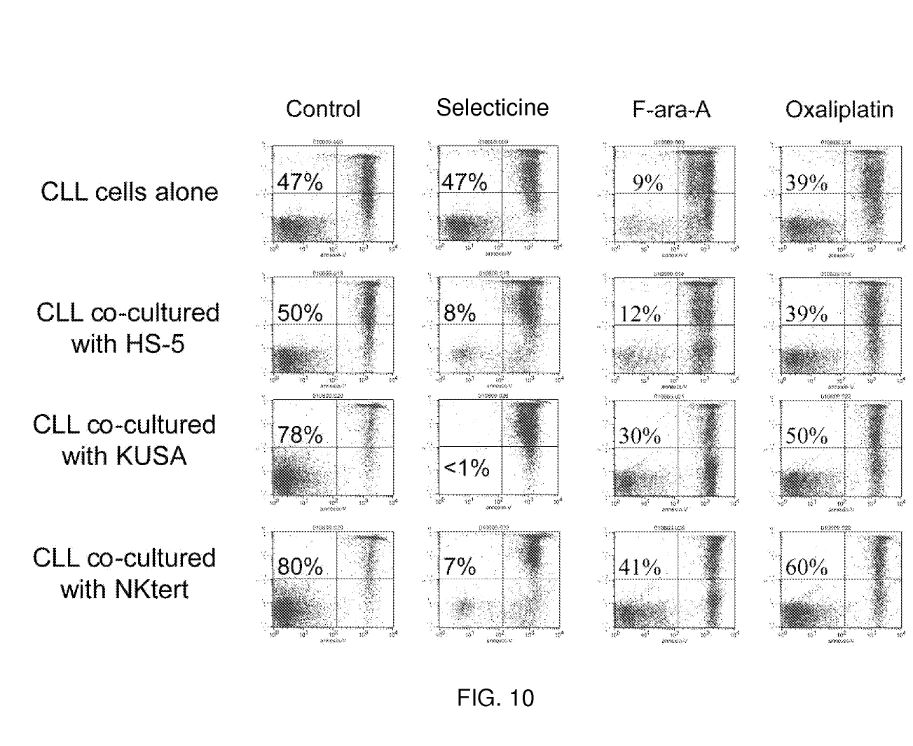

FIG. 10 provides comparison of cytotoxicity of selecticine, Fara-A, and oxaliplatin in CLL cells, cultured alone or in the presence of different types of stromal cells. CLL cells were isolated from patient #10, and cultured in vitro alone or in the presence of different types of stromal cells as indicated. The cells were then treated with 50 μM selecticine, 20 μM F-ara-A, or 20 μM oxaliplatin for 2 days. Cell viability was then measured by flow cytometry analysis after double-staining with annexin-V and PI. The numbers indicate % of viable cells. This data shows that stromal cells protected CLL from the cytotoxicity of standard anti-CLL drugs (F-ara-A and oxalipilatin) but sensitized CLL cells to selecticine.

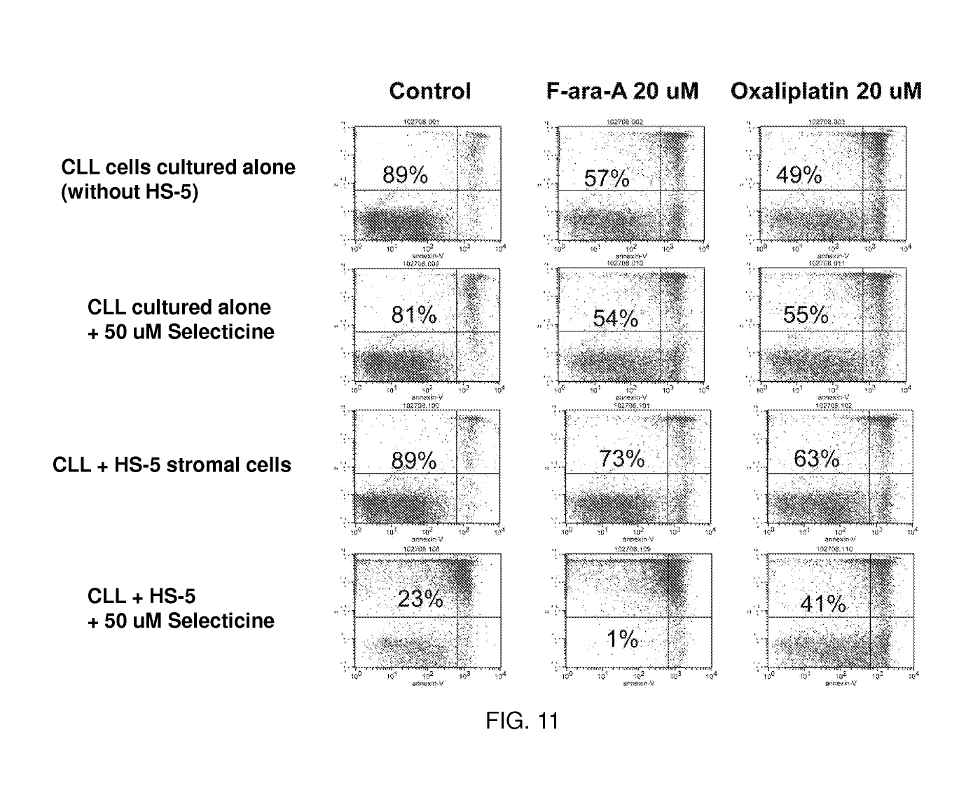

FIG. 11 shows enhancement of cytotoxic action of fludarabine and oxaliplatine by selecticine in CLL cells co-cultured with stroma cells. CLL cells were isolated from patient #11, and cultured alone or in the presence of human bone marrow stromal cells HS-5 as indicated. The cells were then treated with 20 μM F-ara-A or 20 μM oxaliplatin in the presence or absence of selenocystine as indicated for 2 days. Cell viability was then measured by flow cytometry analysis after double-staining with annexin-V and PI. The numbers indicate % of viable cells.

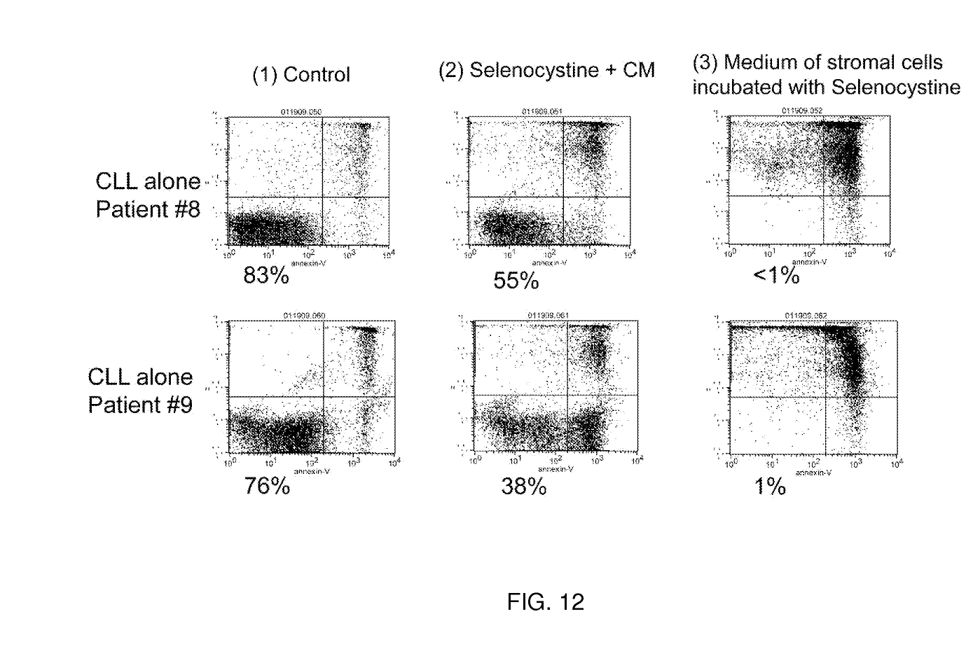

In FIG. 12, it is shown that there is a stromal factor secreted in the culture medium that can convert selenocystine into an active compound that is highly toxic to CLL cells.

CLL cells isolated from two different patients were treated as follow: (1) Control (CLL alone); (2) Selenocystine mixed with the conditioned medium (CM) of stromal cell culture; (3) Medium from stromal cell culture incubated with selenocystine. After an additional 48-h incubation with CLL cells (without stramal), cell viability was measured by flow cytometry analysis after double-staining with annexin-V and PI. The numbers indicate % of viable cells.

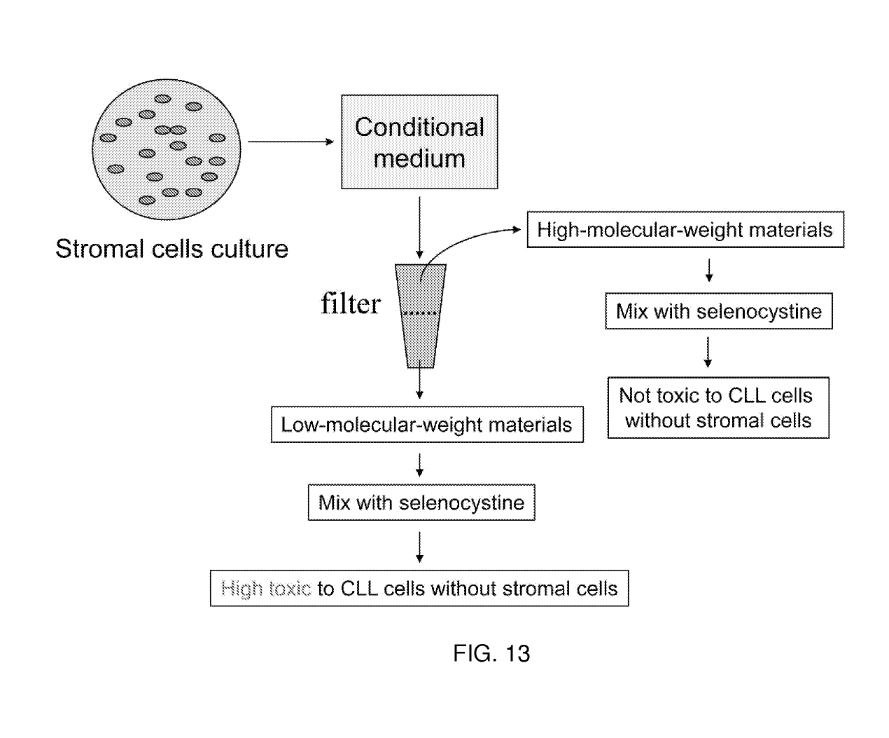

FIG. 13 illustrates exemplary procedures to identify a low-molecular-weight factor that converts selenocystine to an active compound selectively toxic to CLL cells.

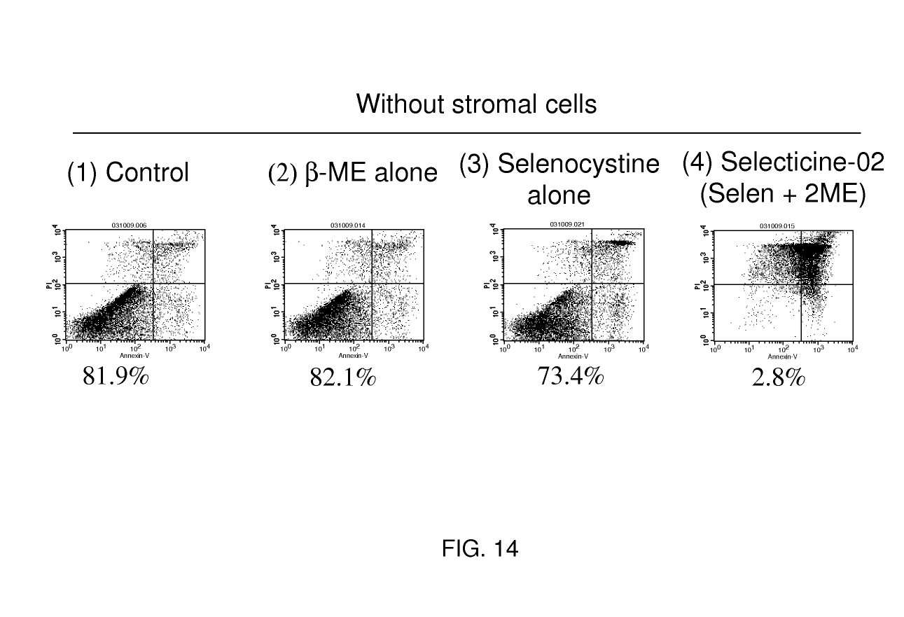

In FIG. 14, selecticine-02 is highly toxic to CLL cells, even in the absence of stroma cells. Selecticine-02 was prepared at a ratio of 1:4 (selenocystine:2-marcaptoethanol). CLL cells (without stromal) were then incubated for 72 hours with: (1) control medium; (2) 200 μM 2-mercaptoethanol (2ME) alone; (3) 50 μM selenocystine alone; (4) Selecticine-02 (50 μM selenocystine+200 μM 2-mercaptoethanol pre-mixed overnight). Cell viability was measured by flow cytometry analysis after double-staining with annexin-V and PI. The numbers indicate % of viable cells. Note: in the presence of stromal cells, selecticine is also extremely effective in killing CLL cells.

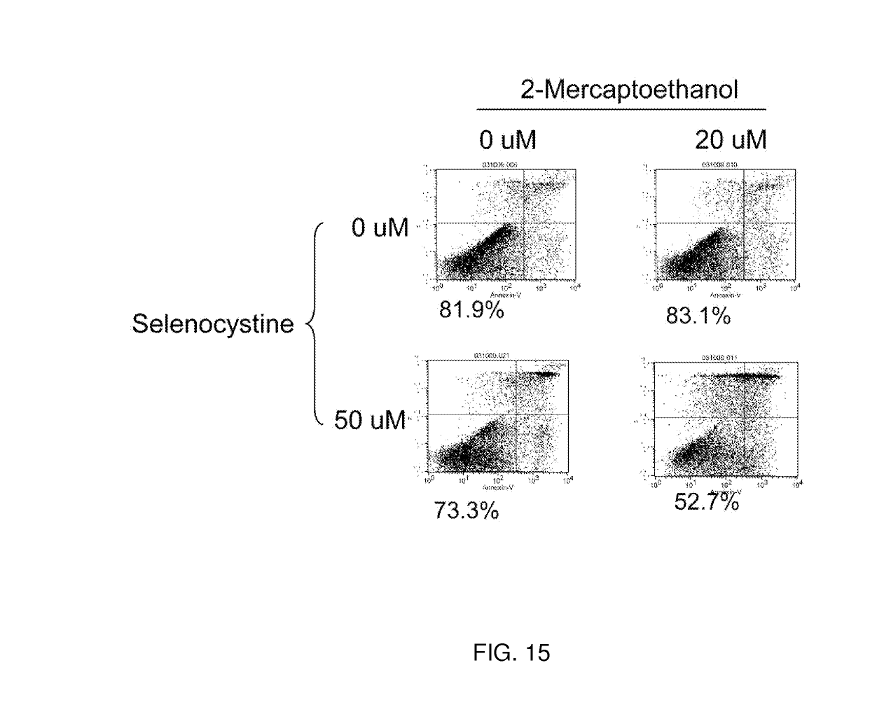

FIG. 15 demonstrates the molar ration of selenocystine:2-mercaptoethanol is critical for the cytotoxic activity of Selecticine-02. A mixture of selenocystine and 2-mercaptoethanol (2ME) at the ration of 5:2 was not sufficient to generate potent cytotoxicity against CLL cells without stromal. The experimental conditions were the same as FIG. 14, except the selenocystine:2ME ration was 5:2 instead of 1:4. CLL cells were from the same patient. Thus, higher molar concentration of 2ME is important for the efficacy of Selecticine-02.

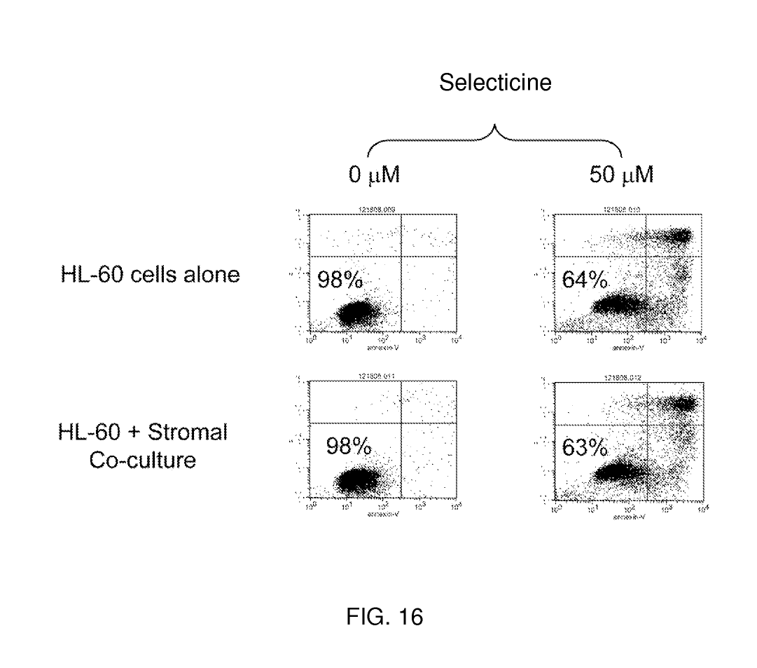

FIG. 16 shows that selecticine exhibited moderate cytotoxicity in human promyelocytic leukemia cells (HL-60) in the presence or absence of stromal cells. HL-60 cells were cultured alone or with human bone marrow stromal cells (HS-5 cell line) as indicated. The cells were then treated with 50 μM selecticine, and cell viability was measured by flow cytometry analysis after double-staining with annexin-V and PI. The numbers indicate % of viable cells. The inability of stromal cells to enhance the cytotoxicity of selenocystine in HL-60 cells is in contrast with that observed in CLL cells, which undergo massive cell death when treated with 50 μM selecticine in the presence of stromal cells.

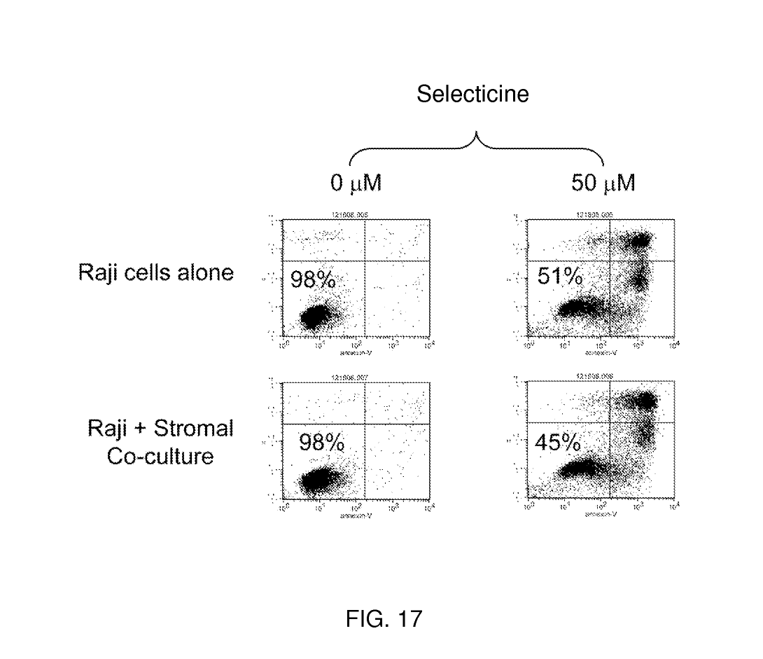

FIG. 17 demonstrates that selecticine exhibits cytotoxic effect on human B cell lymphoma cells (Raji) and the presence of stromal cells further increased this cytotoxic effect. Raji cells were cultured alone or in the presence of human bone marrow stromal cells (HS-5 cell line) as indicated. The cells were then treated with 50 μM selenocystine, and cell viability was measured by flow cytometry analysis after double-staining with annexin-V and PI. The numbers indicate % of viable cells.

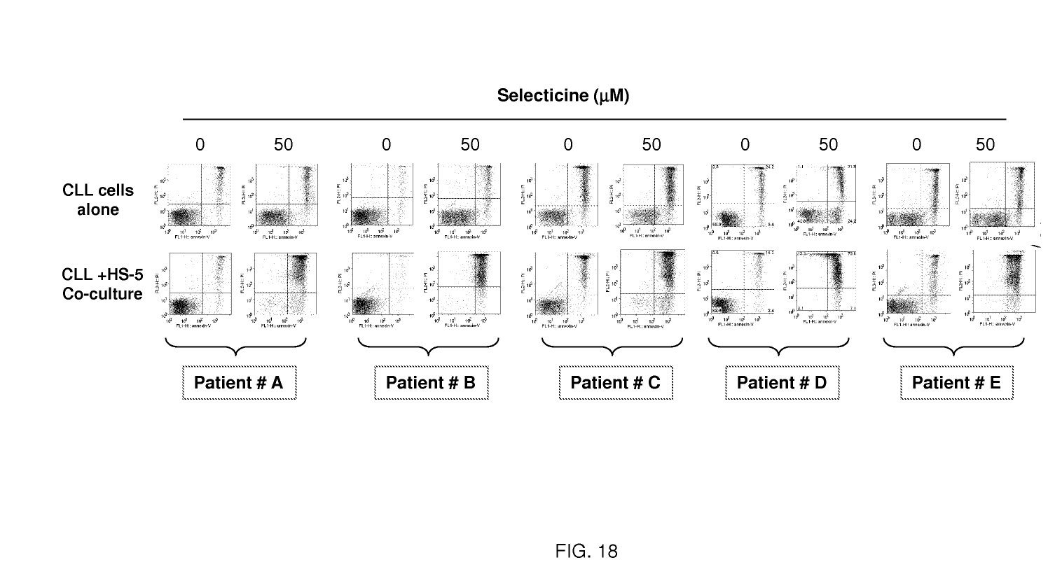

FIG. 18 shows selective killing of primary leukemia cells isolated from multiple CLL patients by selecticine in the presence of stromal cells. CLL cells were isolated from patients A-E, and cultured in vitro alone or in the presence of human bone marrow stromal cells (HS-5 cells) as indicated. The cells were then treated with 50 mM selenocystine for 2 days. Cell viability was then measured by flow cytometry analysis after double-staining with annexin-V and PI.

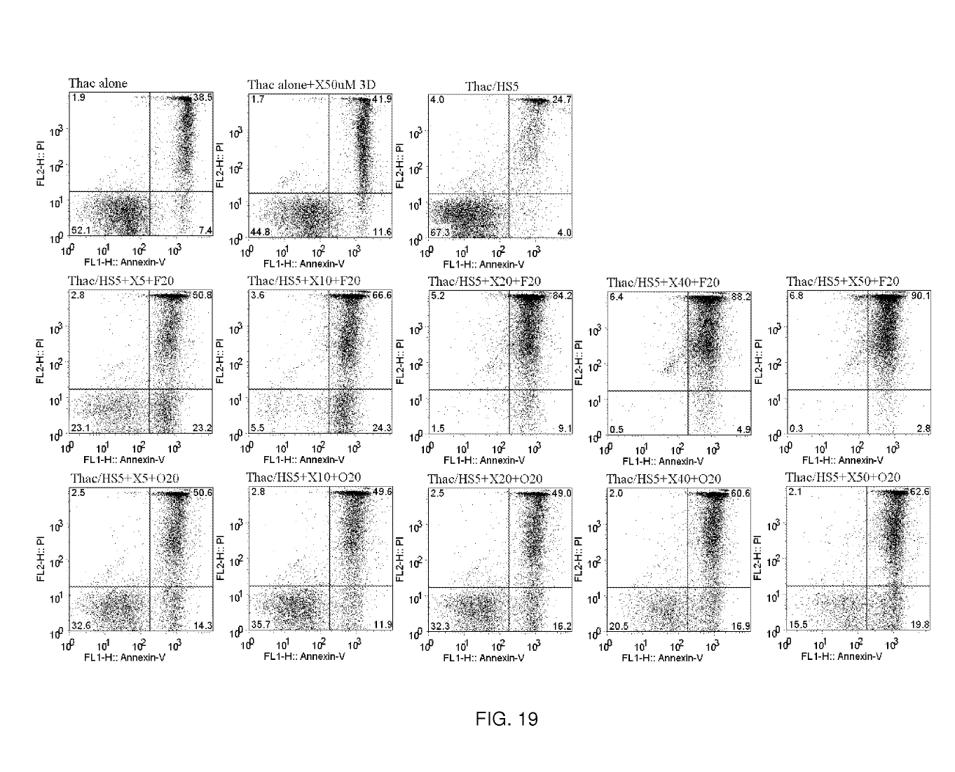

FIG. 19 provides that selecticine enhances the cytotoxicity of fludarabine and oxaliplatine in CLL cells co-cultured with HS-5 stromal cells. CLL cells were isolated from patient #12, and cultured alone or in the presence of human bone marrow stromal cells (HS-5). The cells were then treated with 20 μM F-ara-A (F) or 20 mM oxaliplatin (O) in the presence or absence of 5, 10 or 40 M selecticine (X) as indicated. Cell viability was then measured by flow cytometry analysis after double-staining with annexin-V and PI.

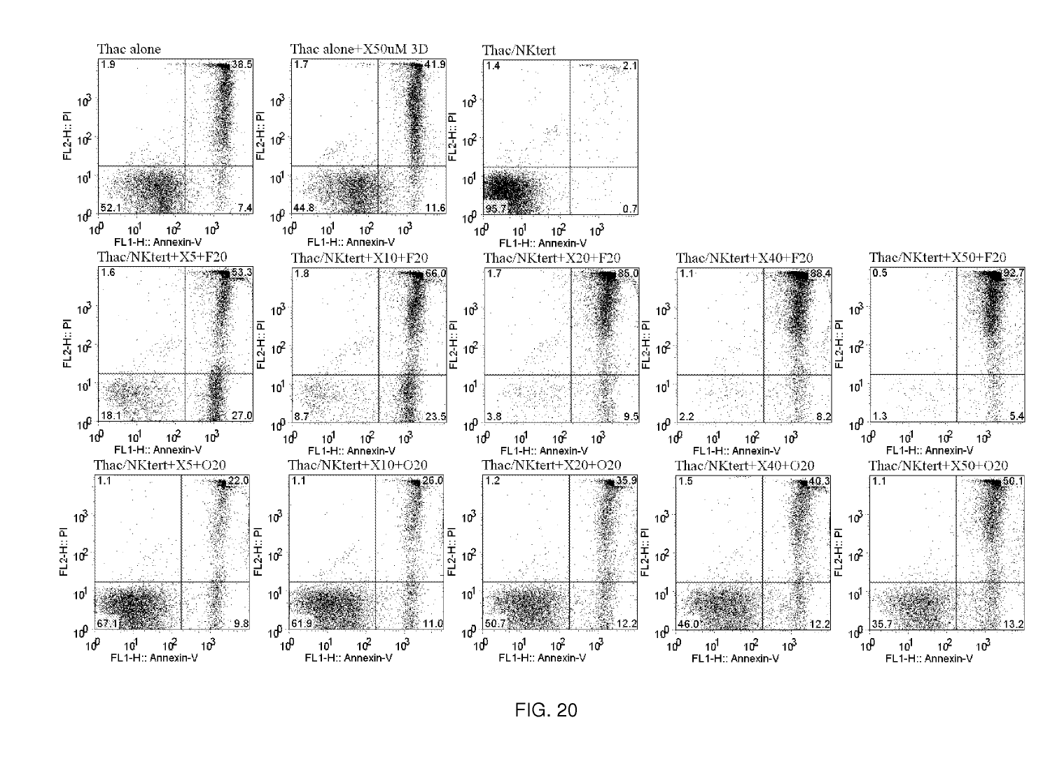

FIG. 20 shows selecticine enhances the cytotoxicity of fludarabine and oxaliplatine in CLL cells co-cultured with NKtert stromal cells. CLL cells were isolated from patient #12, and cultured alone or in the presence of NKtert stromal cells. The cells were then treated with 20 μM F-ara-A (F) or 20 μM oxaliplatin (O) in the presence or absence of 5, 10 or 40 μM selecticine (X) as indicated. Cell viability was then measured by flow cytometry analysis after double-staining with annexin-V and PI.

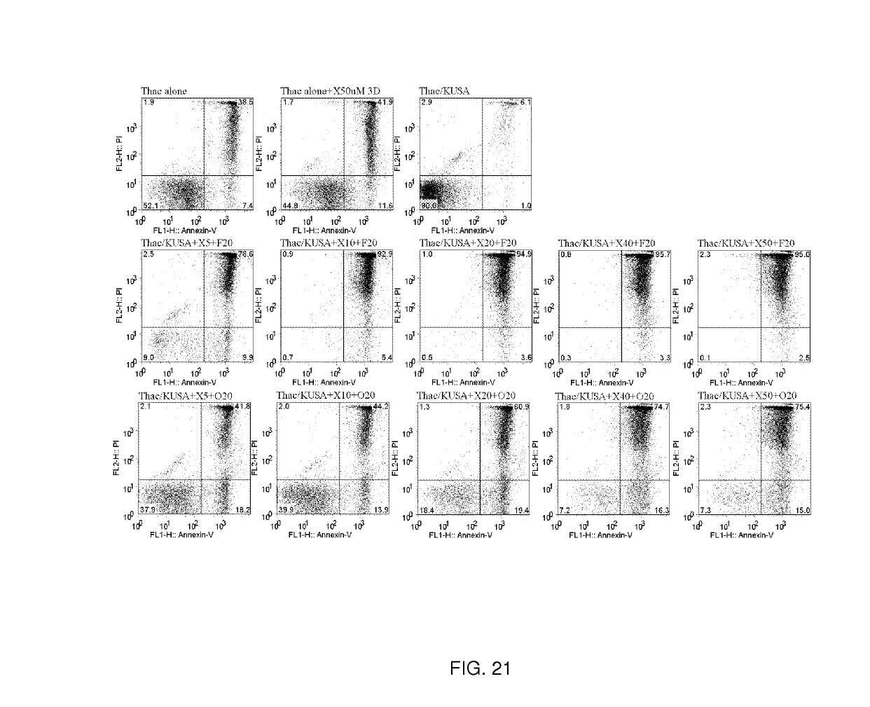

FIG. 21 demonstrates that selecticine enhances the cytotoxicity of fludarabine and oxaliplatine in CLL cells co-cultured with KUSA stromal cells. CLL cells were isolated from patient #12, and cultured alone or in the presence of KUSA stromal cells. The cells were then treated with 20 μM F-ara-A (F) or 20 μM oxaliplatin (O) in the presence or absence of 5, 10 or 40 μM selecticine (X) as indicated. Cell viability was then measured by flow cytometry analysis after double-staining with annexin-V and PI.

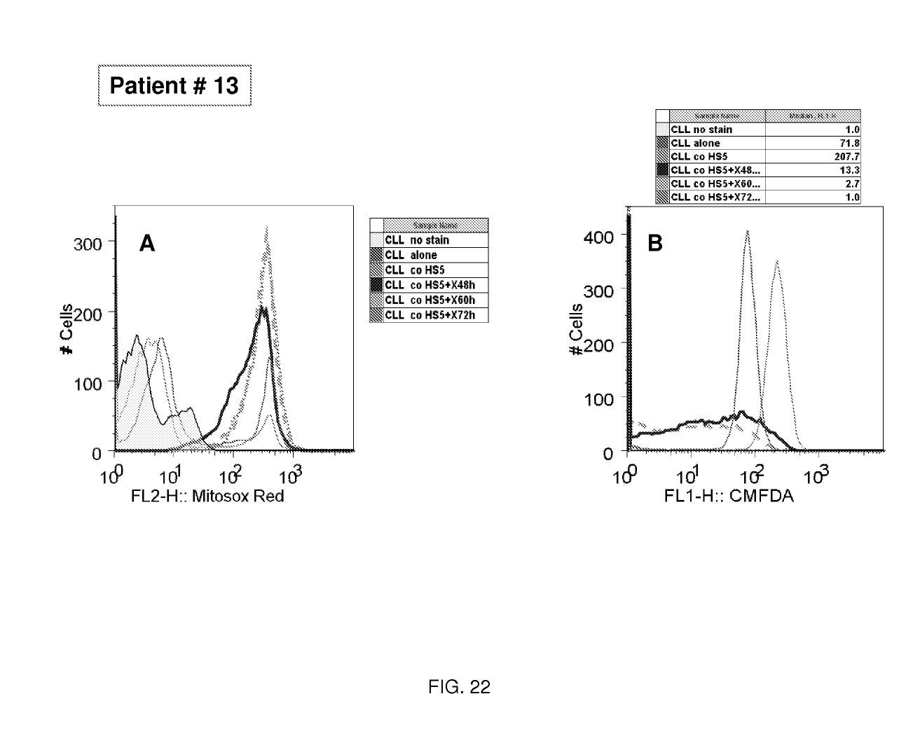

FIG. 22 shows that selecticine promotes ROS generation and causes depletion of cellular thiol in CLL cells in the presence of stromal cells. CLL cells were incubated with 50 μM selecticine in the presence or absence of stromal cells (HS-5) as indicated. Mitochondrial ROS levels were stained by mitosox Red (A) and cellular thiol was stained by CMFDA (B). Analysis of cellular glutathione showed that Selecticine caused severe depletion of GSH in CLL cells.

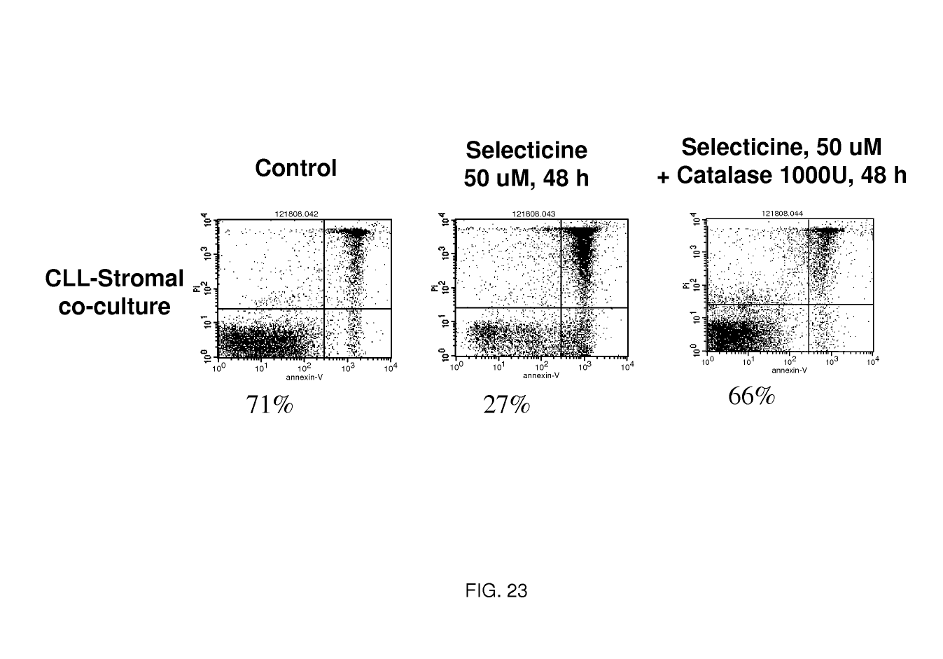

FIG. 23 demonstrates that catalase abrogated the cytotoxic effect of selecticine in CLL cells under stromal co-culture conditions. CLL cells were isolated from patient #14, and cultured in the presence of human bone marrow cells. The cells were then treated with 50 μM selecticine in the presence or absence of catalase as indicated. Cell viability was measured by flow cytometry analysis after double-staining with annexin-V and PI. The numbers indicate % of viable cells.

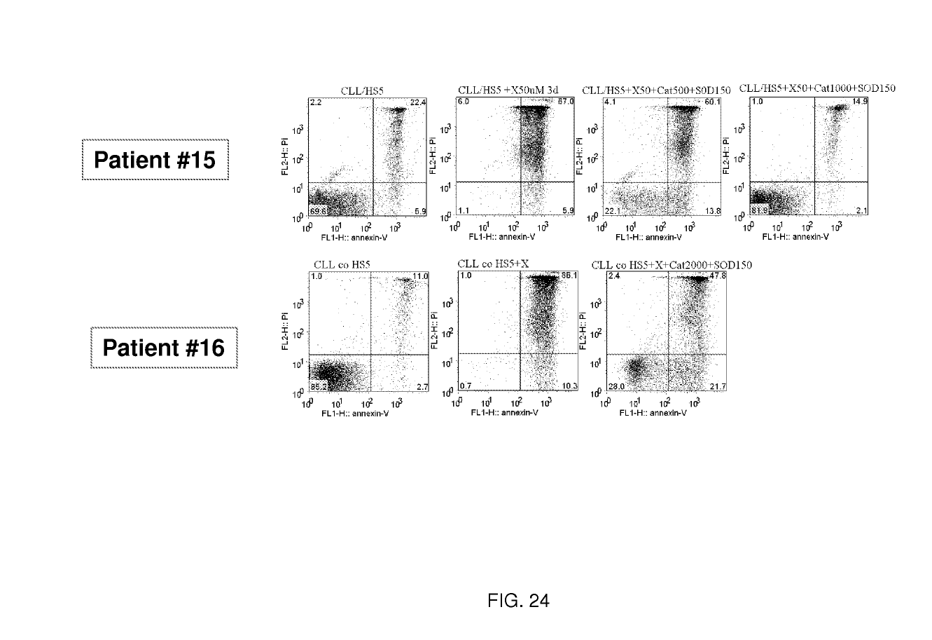

FIG. 24 shows that SOD and catalase protect CLL cells from the cytotoxic effect of selecticine in the presence of stromal cells. CLL cells were isolated from patients #15 and #16, and cultured in the presence of human bone marrow cells. The cells were then treated with 50 μM selecticine (X) in the presence or absence of SOD1 and catalase (Cat) as indicated. Cell viability was measured by flow cytometry analysis after double-staining with annexin-V and PI.

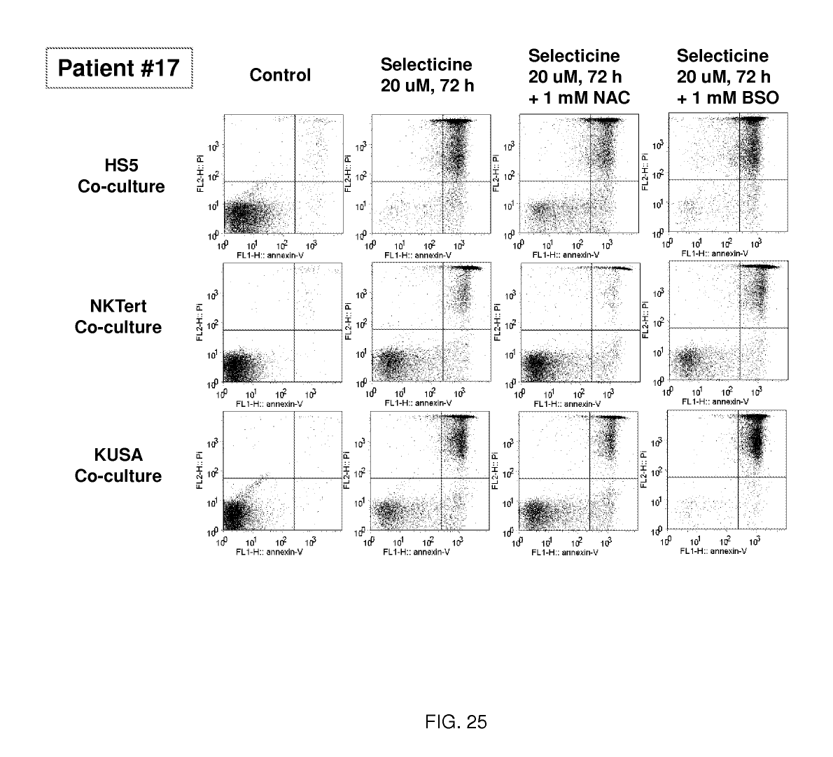

FIG. 25 shows the effect of antioxidant NAC and the glutathione-depleting agent BSO on the cytotoxic effect of selecticine in the presence of stromal cells. CLL cells were isolated from patient #17, and co-cultured with various types of marrow cells as indicated. The cells were then treated with 20 μM selecticine in the presence or absence of NAC or BSO as indicated. Cell viability was measured by flow cytometry analysis after double-staining with annexin-V and PI. The results showed that the antioxidant NAC protected CLL against toxicity of selecticine, while depletion of GSH by BSO enhanced the toxicity.

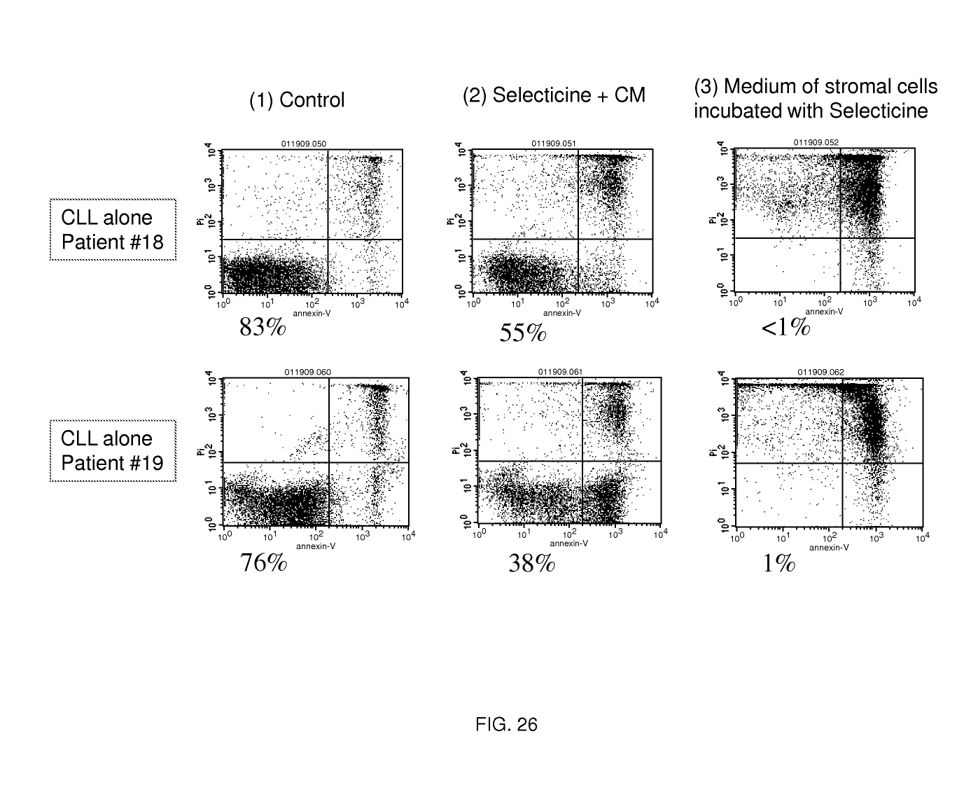

FIG. 26 shows a stromal factor secreted in the culture medium can convert selecticine into an active compound that is highly toxic to CLL cells. CLL cells isolated from two different patients were treated as follow: (1) Control (CLL alone). (2) Selecticine mixed with the conditioned medium (CM) of the control stromal cell culture. (3) Medium from the stromal cell culture incubated with selenocystine. After an additional 48-h incubation with CLL cells (without stromal cells), cell viability was measured by flow cytometry analysis after double-staining with annexin-V and PI. The numbers indicate % of viable cells.

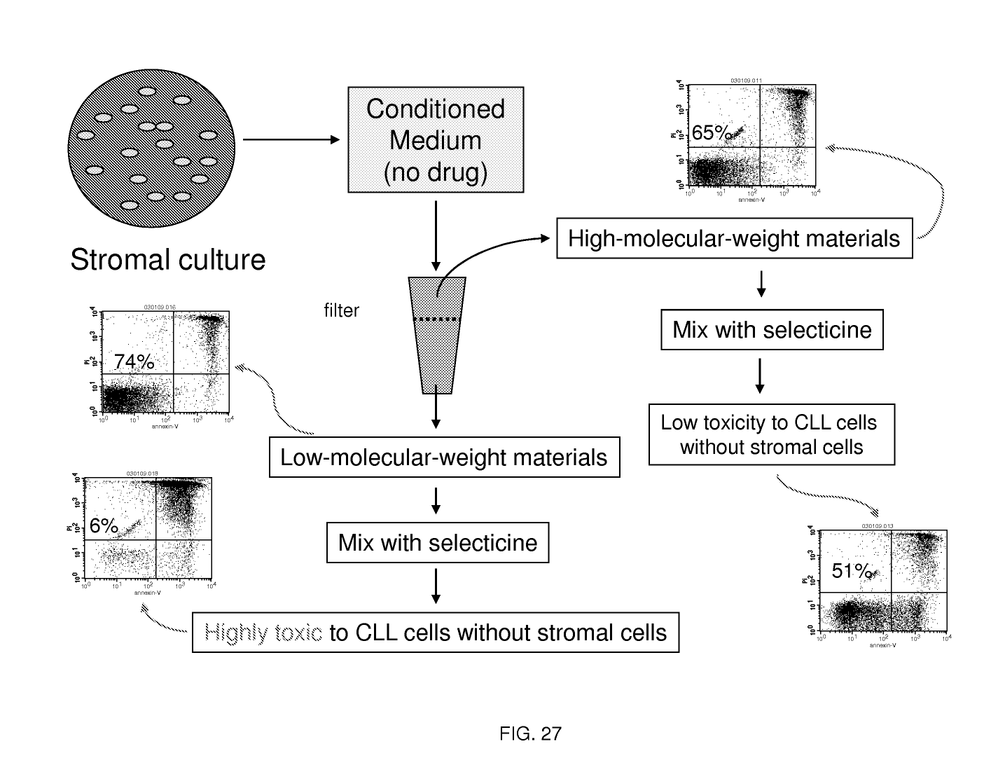

FIG. 27 illustrates exemplary procedures to identify a low-molecular-weight factor that converts selecticine to an active compound selectively toxic to CLL cells. CLL cells were incubated with the indicated medium fractions for 48 h, and cell viability was measured by flow cytometry analysis after double-staining with annexin-V and PI. The numbers indicate % of viable cells.

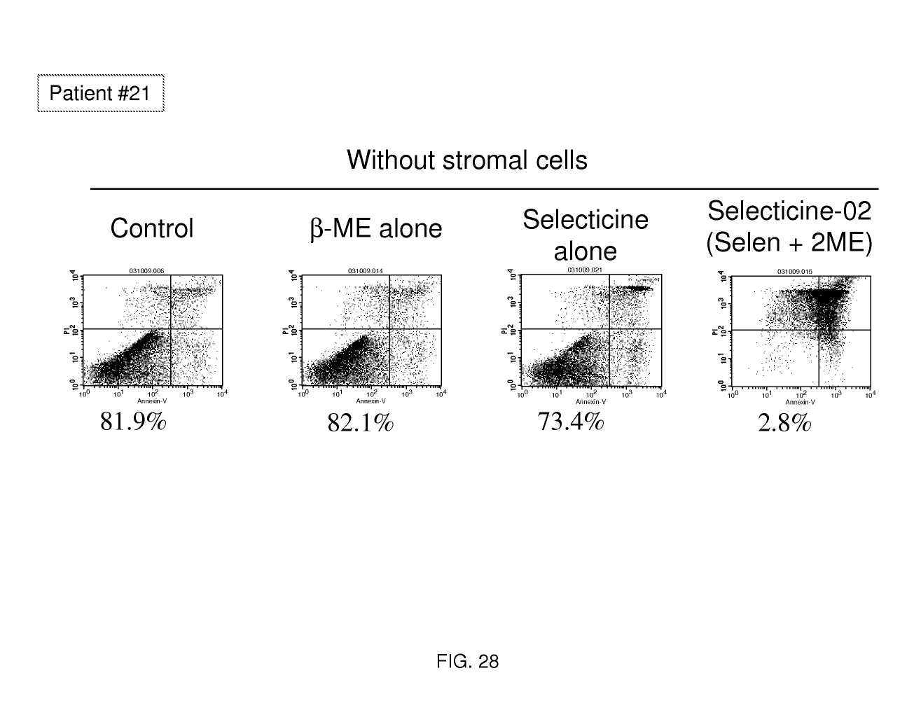

FIG. 28 shows selecticine-02 is highly toxic to CLL cells, even in the absence of stroma cells. Selecticine-02 was prepared at a ratio of 1:4 (selenocystine: β-mercaptoethanol). CLL cells (without stromal) were then incubated for 72 hours with: (1) control medium; (2) 200 μM β-mercaptoethanol ((β-ME) alone; (3) 50 μM selecticine; (4) 50 μM Selecticine-02. Cell viability was measured by flow cytometry analysis after double-staining with annexin-V and PI. The numbers indicate % of viable cells. Note that selecticine was ineffective in the absence of stromal cells, while selecticine-02 is highly active against CLL cells without stromal cells.

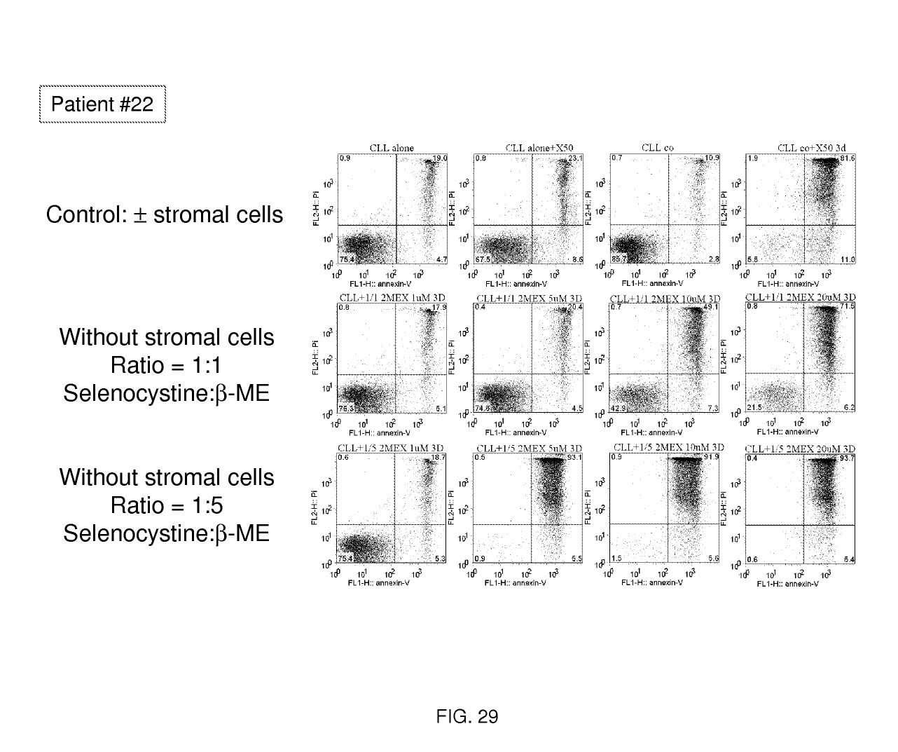

FIG. 29 demonstrates the ration of selenocystine: β-mercaptoethanol is critical for the activity of selecticine-02. The ratio of 1:5 (selenocystine: β-mercaptoethanol) was highly active, while a ratio of 1:1 only produced modest activity. Cell cells were incubated with selecticine in the presence or absence of stromal cells, or incubated with the indicated concentrations (5-20 uM) of Selecticine-02 with different ratios of selenocystine: β-mercaptoethanol without stromal cells for 3 days. Cell viability was measured by flow cytometry analysis.

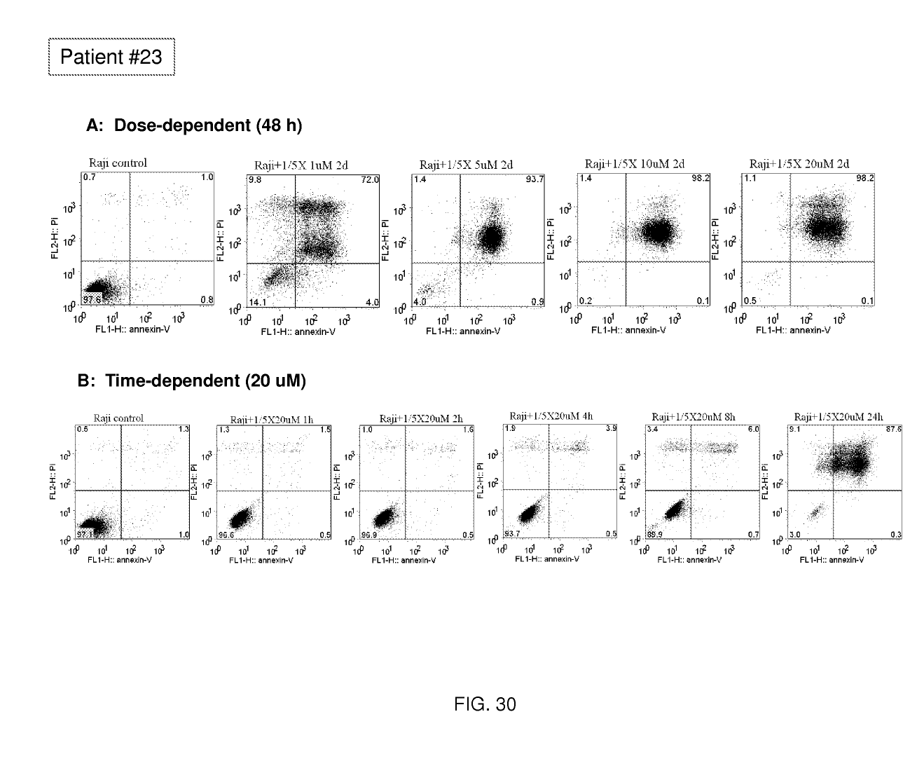

FIG. 30 shows that selecticine-02 with a ration of 1:5 (selenocystine: β-mercaptoethanol) was highly effective against B-cell lymphoma. B-lymphoma cells (Raji) were incubated with the indicated concentrations of selecticine-02 with a 1:5 ratio of selenocystine: β-mercaptoethanol for the indicated times. Cell viability was measured by flow cytometry analysis.

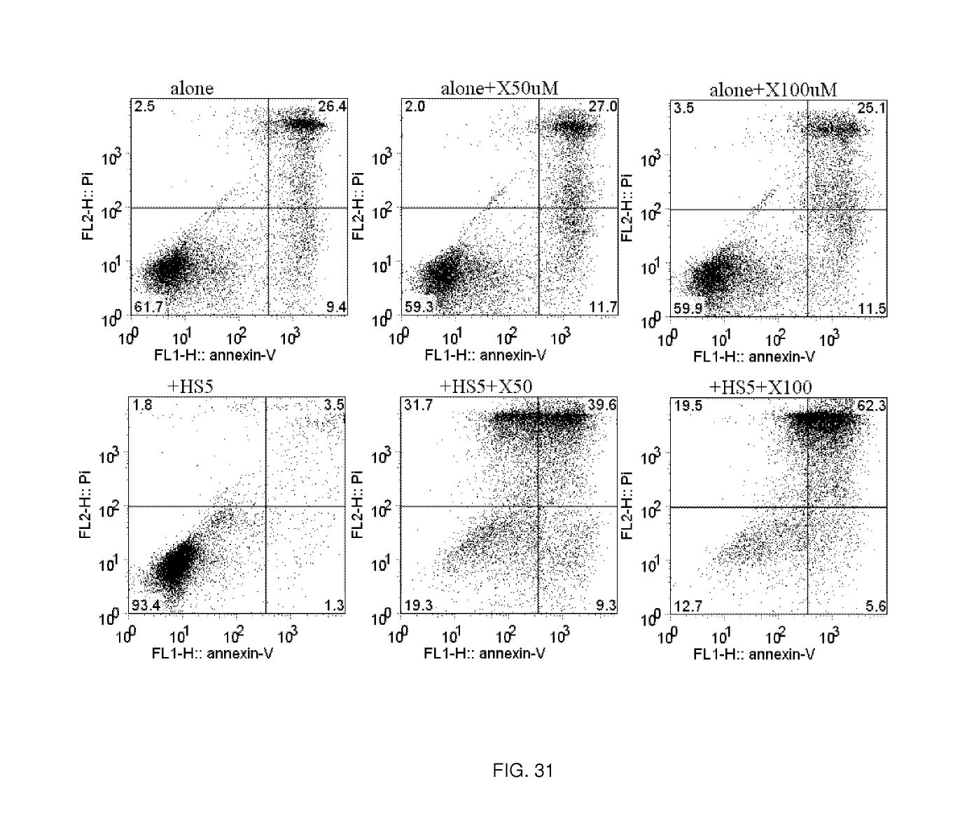

FIG. 31 shows selecticine effectively killed primary leukemia cells from a patients with acute myeloid leukemia (AML) in the presence of bone marrow stromal cells. Primary AML cells were cultured alone or in the presence of human bone marrow stromal cells (HS-5 cell line) as indicated. The cells were then treated with 50-100 μM selecticine, and cell viability was measured by flow cytometry analysis after double-staining with annexin-V and PI. The numbers indicate % of viable cells.

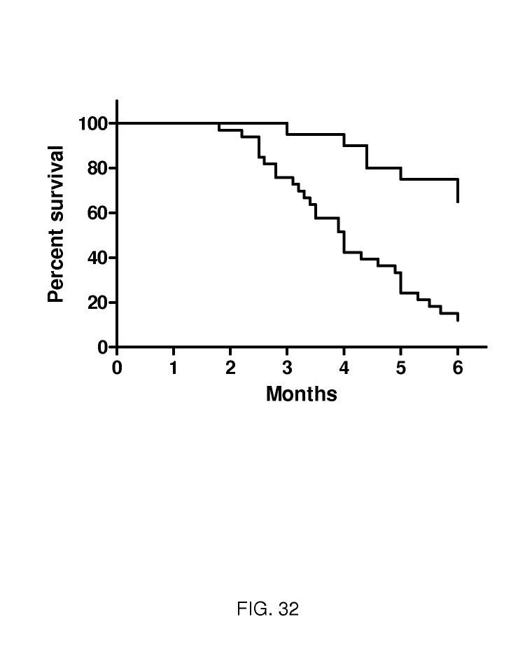

FIG. 32 presents in vivo therapeutic activity of Selecticine in CLL mouse model. Mice with TCL-1 transgenic/p53−/− genotype develop develop aggressive CLL disease and start to die at age of 2 months, with a median survival time of 4 months. Treatment with Selecticine (3 mg/kg, twice per week M/F, i.v.) was highly effective and significantly improved the overall survival of the mice.

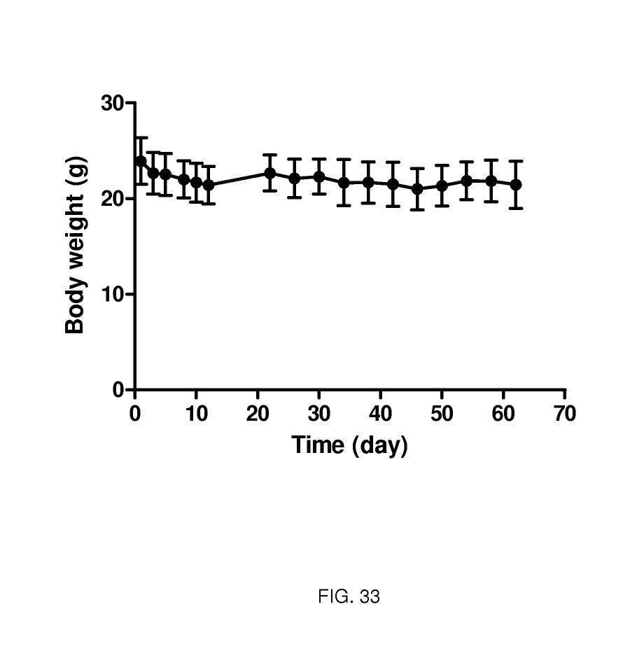

FIG. 33 shows the effect of selecticine treatment on mouse body weights. Mice with TCL-1 transgenic/p53−/− genotype (aggressive CLL disease model) were treated with Selecticine (3 mg/kg, twice per week M/F, i.v.), and mouse body weights were measured.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of treating chronic lymphocytic leukemia in an individual, comprising the step of administering a therapeutically effective amount of a selecticine to the individual.

2. The method of claim 1, wherein the selecticine is selected from the group consisting of selenocystine, selenocysteine, a combination of selenocystine and 2-mercaptoethanol, a combination of selenocystine and dithiothreitol, and a combination of selenocystine and hydrochloric acid.

3. The method of claim 2, wherein the selecticine is selonocystine.

4. The method of claim 2, wherein the selecticine is selenocysteine.

5. The method of claim 2, wherein the selecticine is a combination of selenocystine and 2-mercaptoethanol.

6. The method of claim 2, wherein the selecticine is a combination of selenocystine and hydrochloric acid.

7. The method of claim 2, wherein the selecticine is a combination of selenocystine and dithiothreitol.

8. The method of claim 1, further comprising administering an additional anti-cancer therapy to the individual.

9. The method of claim 8, wherein the anti-cancer therapy comprises chemotherapy, immunotherapy, surgery, stem cell transplant, or radiation.

10. The method of claim 9, wherein the chemotherapy comprises fludarabine, cyclophosphamide, bendamustine and/or oxaliplatin.

11. The method of claim 9, wherein the immunotherapy comprises rituximab, alemtuzumab, ofatumumab, cyclophosphamide, lumiliximab, and/or lenalidomide.

12. The method of claim 1, wherein the selecticine is delivered to the individual intravenously or orally.

13. The method of claim 1, further comprising the step of diagnosing chronic lymphocytic leukemia.
